(19) United States
(12) Patent Application Publication
(10) Pub. No.: US 2023/0390478 A1
(43) Pub. Date: Dec. 7, 2023

(54) SYSTEMS AND METHODS FOR DETERMINING USAGE OF BALANCED SALT SOLUTION IN AN OPHTHALMIC SURGICAL SYSTEM

(71) Applicant: Johnson & Johnson Surgical Vision, Inc., Irvine, CA (US)

(72) Inventors: Edith Fung, Diamond Bar, CA (US); Deep Mehta, Irvine, CA (US); Abraham Hajishah, Irvine, CA (US); Jianbo Zhou, Irvine, CA (US); Dung Ma, Garden Grove, CA (US)

(21) Appl. No.: 18/325,913

(22) Filed: May 30, 2023

Related U.S. Application Data
(60) Provisional application No. 63/365,705, filed on Jun. 1, 2022.

(51) Int. Cl. A61M 1/00

(52) U.S. Cl. CPC A61M 1/777; A61M 1/72; A61M 2205/18; A61M 2205/3334; A61M 2210/0612

(57) ABSTRACT

The present aspect of the disclosure uses software to monitor the fluid amount used and available to a surgical system and may inform a user when the available fluid approaches a depletion threshold so a user may take appropriate steps to minimize impact to the surgical operation.

This application claims priority to U.S. Provisional Patent Application No. 63/365,705, filed on Jun. 1, 2022, the entire contents of which are hereby incorporated by reference.

### FIELD OF THE INVENTION

The present disclosure relates to phacoemulsification fluidics system control, and, more particularly a system and method for determining usage of balanced salt solution in an ophthalmic surgical system.

### BACKGROUND

Cataracts affect more than 24 million Americans aged 40 and older. Cataract surgery entails the removal of a lens of an eye that has developed clouding of the eye's natural lens, or opacification. As a result of opacification, light is unable to travel to the retina, thereby causing vision loss. Once vision becomes impaired, cataract surgery is a viable option with a high level of success. During cataract surgery, a surgeon replaces the clouded lens with an intraocular lens (IOL).

Certain surgical procedures, such as phacoemulsification surgery, have been successfully employed in the treatment of certain ocular problems, such as cataracts. Phacoemulsification surgery utilizes a small corneal incision to insert the tip of at least one phacoemulsification handheld surgical implement, or handpiece, through the corneal incision. The handpiece includes a needle which is ultrasonically driven once placed within the incision to emulsify the eye lens, or to break the cataract into small pieces. The broken cataract pieces or emulsified eye lens may subsequently be removed using the same handpiece, or another handpiece, in a controlled manner. The surgeon may then insert a lens implant into the eye through the incision. The incision is allowed to heal, and the result for the patient is typically significantly improved eyesight.

During the phacoemulsification process for cataract removal, the system utilizes the ultrasound power to break and dispose the cataract. The surgical process involves a fluidics system where balanced salt solution) from a source (e.g., bottle or bag) is delivered to the anterior chamber of the eye via an irrigation port located on the handpiece. The cataract particles and fluid are aspirated to a disposable bag using a pump system, which may include one or more pumps, e.g., vacuum based and/or flow-based pumps. The fluid inflow and outflow are managed by the system in order to maintain the pressure in the anterior chamber. The problem with existing systems using either a balanced salt solution bottle or bag is that the systems cannot detect when the balanced salt solution bottle is empty. While focusing on the surgery, the surgeon or operating room (OR) staff cannot constantly monitor the balanced salt solution level in the bottle. This could cause a loss of pressure in the anterior chamber if the balanced salt solution source runs out of fluid.

### BRIEF SUMMARY OF THE INVENTION

The present aspect of the disclosure uses software to monitor the balanced salt solution level available to a surgical console and may inform a user (e.g., surgeon or nurse) when the available balanced salt solution is approaching the empty level so the user may take appropriate steps to minimize impact to the surgical operation.

The present aspect of the disclosure provides for a surgical system, comprising a system for monitoring the amount of fluid used during a phacoemulsification surgical system, comprising a fluid source connected to a surgical console having a known volume of balanced salt solution, a cassette having a known fluid capacity, an encoder for tracking the activity of a drainage pump in communication with the cassette, and an irrigation valve control providing a plurality of state indications. The amount of balanced salt solution removed from the fluid source may be calculated by summing the amount of fluid associated with the drainage pump activity, the plurality of state indications and the fluid capacity of the cassette. Each state indication is indicative of an open or closed valve position and the activity of the drainage pump may be selected from the group consisting of pump runtime, pump speed, and direction.

The present aspect of the disclosure provides a method for calculating the amount of fluid used in a phacoemulsification surgical system. The present aspect of the disclosure provides for the calculating of the amount of balanced salt solution usage by the surgical console, wherein the instrument host receives values for the fluid capacity of a surgical cassette, the external release of balanced salt solution, the measuring of drainage activity by the drainage pump encoder, and the plurality of state indications for an irrigation valve control. The amount of balanced salt solution usage by the surgical console may be calculated by subtracting from the received supply value of balanced salt solution the sum of the amount of fluid associated with each of the instrument host received values, and sending to a display of the console an indication of the calculated amount of BBS usage.

### BRIEF DESCRIPTION OF THE DRAWINGS

This disclosure is illustrated by way of example and not by way of limitation in the accompanying figure(s). The figure(s) may, alone or in combination, illustrate one or more examples of the disclosure. Elements illustrated in the figure(s) are not necessarily drawn to scale. Reference labels may be repeated among the figures to indicate corresponding or analogous elements.

The detailed description refers to the accompanying figures in which:

FIG. 1A is a schematic illustrating an eye treatment system in which a cassette is coupled to an eye treatment probe with an eye treatment console under one example;

FIG. 1B is a schematic illustrating a surgical eye treatment console under another example of an aspect of the disclosure;

FIG. 2 is a schematic illustrating a cup fill example of an aspect of the disclosure;

FIG. 3 is a schematic illustrating a continuous irrigation example of an aspect of the disclosure;

FIG. 4 is a schematic illustrating a continuous irrigation example of an aspect of the disclosure;

FIG. 5 is a schematic illustrating an aspect of the disclosure;

FIG. 6 is a schematic illustrating an aspect of the disclosure;

FIG. 7 is a schematic illustrating a surgery example of an aspect of the disclosure;

FIG. 8 is a schematic illustrating a prime/tune example of an aspect of the disclosure;

FIGS. 9A-9D is a flow diagram illustrating a balanced salt solution tracking algorithm of an aspect of the disclosure;

FIGS. 10A-10B is a flow diagram illustrating a warning system when the balanced salt solution is below a threshold value;

FIG. 11 is a flow diagram illustrating a balanced salt solution tracking algorithm of an aspect of the disclosure;

FIG. 12 is an exemplary graphical user interface (GUI);

FIG. 13 is an exemplary GUI; and

FIG. 14 is an exemplary GUI.

### DETAILED DESCRIPTION

The figures and descriptions provided herein may have been simplified to illustrate aspects that are relevant for a clear understanding of the herein described apparatuses, systems, and methods, while eliminating, for the purpose of clarity, other aspects that may be found in typical similar devices, systems, and methods. Those of ordinary skill may thus recognize that other elements and/or operations may be desirable and/or necessary to implement the devices, systems, and methods described herein. But because such elements and operations are known in the art, and because they do not facilitate a better understanding of the present disclosure, for the sake of brevity a discussion of such elements and operations may not be provided herein. However, the present disclosure is deemed to nevertheless include all such elements, variations, and modifications to the described aspects that would be known to those of ordinary skill in the art.

Embodiments are provided throughout so that this disclosure is sufficiently thorough and fully conveys the scope of the disclosed examples to those who are skilled in the art. Numerous specific details are set forth, such as examples of specific components, devices, and methods, to provide a thorough understanding of examples of the present disclosure. Nevertheless, it will be apparent to those skilled in the art that certain specific disclosed details need not be employed, and that exemplary examples may be embodied in different forms. As such, the exemplary examples should not be construed to limit the scope of the disclosure. As referenced above, in some exemplary examples, well-known processes, well-known device structures, and well-known technologies may not be described in detail.

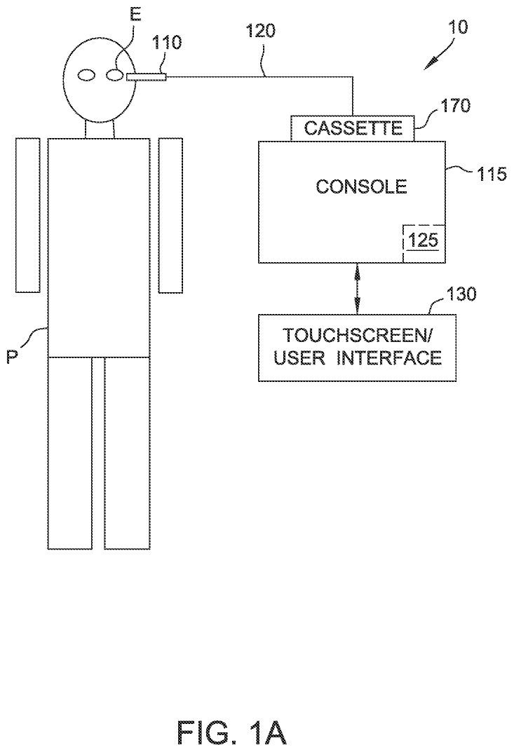

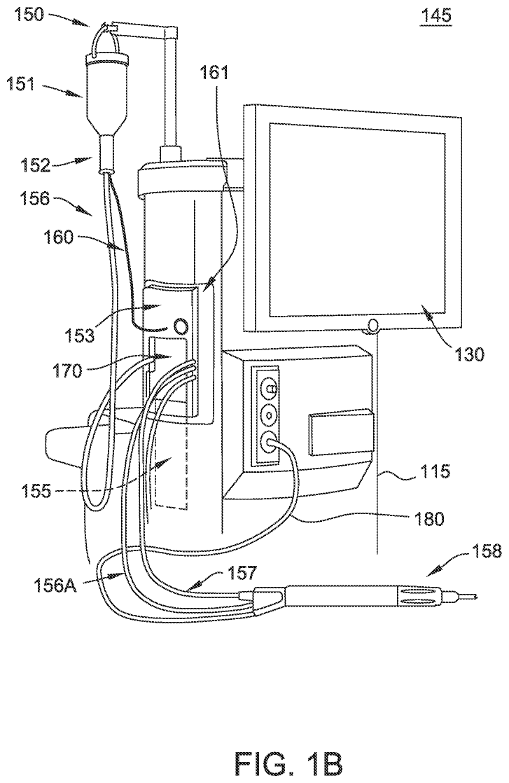

Referring now to FIG. 1A, an eye treatment system 10 for treating an eye E of a patient P generally includes an eye treatment probe handpiece 110 coupled with a console 115 by a cassette 170. Handpiece 110 generally includes a handle for manually manipulating and supporting an insertable probe tip. The probe tip has a distal end which is insertable into the eye, with one or more lumens in the probe tip allowing irrigation fluid to flow from console 115 and/or cassette 170 into the eye. Aspiration fluid may also be withdrawn through a lumen of the probe tip, with console 115 and cassette 170 generally including a vacuum aspiration source, a positive displacement aspiration pump, or both to help withdraw and control a flow of surgical fluids into and out of eye E. As the surgical fluids may include biological materials that should not be transferred between patients, cassette 170 will often comprise a sterilizable (or alternatively, disposable) structure, with the surgical fluids being transmitted through flexible conduits 120 of cassette 170 that avoid direct contact in between those fluids and the components of console 115.

When a distal end of the probe tip of handpiece 110 is inserted into an eye E, for example, for removal of a lens of a patient P with cataracts, an electrical conductor and/or pneumatic line (not shown) may supply energy from console 115 to an ultrasound transmitter of handpiece 110, a cutter mechanism, or the like. Alternatively, handpiece 110 may be configured as an irrigation/aspiration (FA) and/or vitrectomy handpiece. Also, the ultrasonic transmitter may be replaced by other means for emulsifying a lens, such as a high energy laser beam. The ultrasound energy from handpiece 110 helps to fragment the tissue of the lens, which can then be drawn into a port of the tip by aspiration flow. So as to balance the volume of material removed by the aspiration flow, an irrigation flow through handpiece 110 (or a separate probe structure) may also be provided, with both the aspiration and irrigation flows being controlled by console 115.

To avoid cross-contamination between patients without incurring excessive expenditures for each procedure, cassette 170 and its flexible conduits 120 may be disposable. However, the flexible conduit or tubing may be disposable, with the cassette body and/or other structures of the cassette being sterilizable. Cassette 170 may be configured to interface with reusable components of console 115, including, but not limited to, peristaltic pump rollers, a Venturi or other vacuum source, a controller 125, and/or the like.

Console 115 may include controller 125, which may include an embedded microcontroller and/or many of the components common to a personal computer, such as a processor, data bus, a memory, input and/or output devices (including a user interface 130 (e.g., touch screen, graphical user interface (GUI), etc.), and the like. Controller 125 will often include both hardware and software, with the software typically comprising machine readable code or programming instructions for implementing one, some, or all of the methods described herein. The code may be embodied by a tangible media such as a memory, a magnetic recording media, an optical recording media, or the like. Controller 125 may have (or be coupled with) a recording media reader, or the code may be transmitted to controller 125 by a network connection such as an internet, an intranet, an ethernet, a wireless network, or the like. Along with programming code, controller 125 may include stored data for implementing the methods described herein and may generate and/or store data that records parameters corresponding to the treatment of one or more patients.

Referring now to FIG. 1B, a simplified surgical system 145 comprising console 115 is illustrated, where a fluid path may be demonstrated under an exemplary example. In this example, an irrigation source 151 may be configured as a bottle or bag hanging from an IV pole hanger 150. It is understood by those skilled in the art that, while an integrated IV pole is illustrated, other configurations are contemplated, e.g., utilizing standalone/static IV poles, utilizing a pump, pressurized infusion sources, and/or other suitable configurations.

An exemplary irrigation path for fluid may be realized via cassette 170 coupled with cassette interface 153 of the cassette receptacle 161, which receives fluid from irrigation source 151 via drip chamber 152. Irrigation line 156A and aspiration line 157 are coupled with handpiece 158. Irrigation fluid may flow from drip chamber 152 through the irrigation tubing 156 via cassette 170. Irrigation fluid may then flow from the cassette through irrigation line 156A which may be coupled with an irrigation port located on handpiece 158. Aspirated fluid may flow from the eye through the handpiece aspiration line 157 back to cassette 170 and into a waste collection bag 155. Handpiece 158 may be electrically connected to the console 115 by cable 180. Cassette 170 may be removably engaged with console 115 in cassette receptacle 161 which may be shaped to accept only a cassette compatible with console 115. A touch screen display 130 may be provided to display system operation conditions and parameters, and may include a user interface (e.g., touch screen, keyboard, track ball, mouse, remote, etc. —see touchscreen/user interface 130 of FIG. 1A) for entering data and/or instructions to the system of FIG. 1B.

The present aspect of the disclosure provides an algorithm to keep track of the fluid consumed during a surgical procedure and may be software or firmware capable of interfacing with various aspects of a surgical system. More specifically, the present aspect of the disclosure may allow a user of a surgical console to track and calculate balanced salt solution usage before and/or during various points of a surgical procedure, including, cup fill, continuous irrigation, prime/tune, and surgical modes (e.g., Phaco/IA/Vitrectomy). The present aspect of the disclosure employs different techniques to calculate balanced salt solution usage during a variety of system use cases and may make certain assumptions for extra-system losses of fluid, such as wound leakage, for example.

The present aspect of the disclosure may also calculate the amount of fluid used during surgical operation using, for example, the encoder data from a pump associated with either the drain bag or irrigation source. The drained fluid during surgery contributes to the majority of balanced salt solution being consumed. The present aspect of the disclosure may track the starting and ending count of the drain encoder and calculates fluid used from the delta between the two, for example. The present aspect of the disclosure may make certain assumptions on surgical site wound leakage based on the size of tip and sleeve used during surgery. These assumed values may be stored in a lookup table within the console, for example, and may include a time function based on the use of the surgical console. The surgical system may prompt the user to enter the starting volume of the balanced salt solution available to the console during the setup process of the surgical console. The present aspect of the disclosure may use the starting volume of balanced salt solution and the calculated amount of fluid used during a surgical procedure to determine the amount of balanced salt solution remaining through the execution of the many processes that occur with the console during a surgical procedure.

During the cup fill procedure, for example, the present aspect of the disclosure may use the cup fill volume information to calculate the amount of fluid dispensed during the cup fill process. Although a cup fill may be of any predetermined amount, a typical system offers 30 ml, 60 ml and 90 ml for cup fill fluid volume. A user of the surgical console may use continuous irrigation, a method to start and stop dispensing fluid from the balanced salt solution source by opening the irrigation valve (or turning on the irrigation pump) and is controlled through an option in the graphical user interface (“GUI”) software (which together form an irrigation value control), for example, to fill one or more cups prior to prime/tune or the user may use the cup fill feature in place of continuous irrigation to fill a cup. The cup fill rate and time may be based on using the system irrigation tubing with no handpiece attached and with fluid in the tubing. If an attached handpiece is used to fill the cup, the fluid amount dispensed may be less during the time allowed. Similarly, the irrigation tubing should be nearly full to ensure that the amount of cup fill selected by the user on the console, for example, is the amount dispensed.

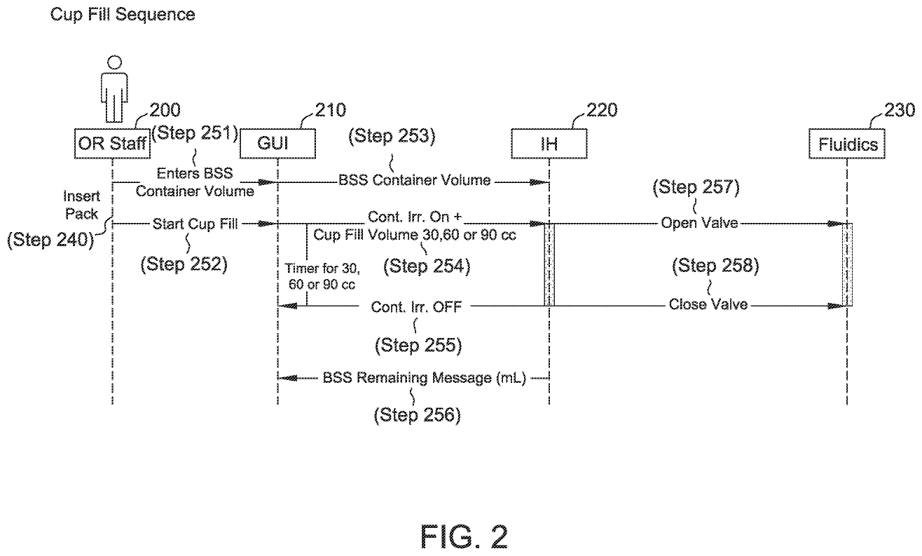

As illustrated in FIG. 2, prior to a surgical procedure, a user 200 may insert a pack (Step 240) and enter into a GUI 210 the volume of balanced salt solution available to the surgical console (Step 251) which may then be recorded to the instrument host 220 (“IH”) (Step 253) which is in communication with various other components of the surgical system which may use the available volume of balanced salt solution. Although any volume amount may be entered, the GUI 210 may prompt for typical volume sizes of balanced salt solution containers which are generally one of 250 ml, 500 ml, 1000 ml, and 2000 ml.

Additionally, the surgical console may carryover the unused balanced salt solution from the previous procedure. For example, if the user started a first procedure with a new 500 ml balanced salt solution bottle, and used 225 ml during the first procedure, at the start of the next procedure, the GUI may indicate that 275 ml is available for use in the current (e.g., second) procedure.

A user may then activate a cup fill sequence (Step 252) through the GUI 210 which may in turn activate an irrigation valve (Step 257) within the fluidics assembly 230 of the console, assuming that the user intends to use the irrigation tubing for the cup fill sequence. The user (200) may select, for example, a cup fill volume of 60 cc which may then activate continuous irrigation (Step 254) for a predetermined time associated with the selected volume. Using this time parameter, the IH 220 may act as the irrigation valve control and will send instructions to the irrigation valve to open for the predetermined time, then close the irrigation valve (Step 258) by turning off the continuous irrigation (Step 255). The IH 220 may also retain a plurality of state indications associated with the irrigation valve control which may provide information related to whether the valve is in an open or closed position or is only partially open, for example. The IH 220 may calculate the amount of balanced salt solution used in the cup fill sequence and will provide to the GUI 210 an indication of the amount of balanced salt solution remaining (Step 256) by subtracting the amount used from the container volume for use with the console.

In an example of the present aspect of the disclosure, the IH 220 may receive an indication of the type of surgical cassette being used with the surgical console and may determine, through a look up table for example, the attributes of the irrigation tubing associated with the cassette which may include, for example, tubing length, diameter, material type, and flow characteristics. The IH 220 may also receive an indication of the balanced salt solution container height, pressure being applied to the balanced salt solution, and/or speed of a pump associated with the irrigation line. Using this information, the IH 220 may calculate the amount of balanced salt solution needed to fill the tubing associated with the inserted surgical cassette prior to any initial actions involving the use of balanced salt solution with the console or IH 220 may have a predetermined constant indicating the amount of fluid used inside a cassette pack once actions started either from priming the cassette pack and/or with the cup fill feature.

By way of nonlimiting example, if in the first console action the user initiates the cup fill sequence, the IH 220 may adjust the time of continuous irrigation to allow for enough balanced salt solution to fill the otherwise empty irrigation tubing of the surgical cassette and then dispense the requested amount of cup fluid, keeping track of each volume used to calculate the amount of balanced salt solution remaining to the console. If, for example, the tubing is already full of balanced salt solution through prior use, then the IH 220 may calculate the amount of balanced salt solution used based on the requested cup fill amount whereas a cup fill for a cassette with empty tubing will require the extra volume to fill the tubing and any other components of the cassette.

Balanced salt solution usage may also be tracked during continuous irrigation by measuring the time elapsed between irrigation valve open and close signals and then calculating the volume dispensed based on time and balanced salt solution container height. The calculation may take into account the type of pack inserted, the aspiration tubing length and/or volume, and inside diameter of the aspiration tubing, tip and/or sleeve size. Generally, identification of the surgical cassette in use with the surgical console will provide the console with the necessary aspiration tubing attributes for calculating fluid use through continuous irrigation, as well as other console functions. Similarly, the console may automatically know or be provided input by the user of the height of the balanced salt solution source to allow for a more precise calculation of fluid flow rate and volume taken from the balanced salt solution source.

In an example of the present aspect of the disclosure, the physical attributes of the cassette pack itself is not calculated, but is a determined to be a constant based on the cassette type. A cassette type constant may be derived by weighing an empty pack (without a drain bag) and then weighing the pack after the surgical case is done (again, without drain bag). The difference in weight between the empty and used pack is determinative of the amount of fluid remaining in the cassette and can be made a constant which may be added to the total amount of balanced salt solution used in the surgical case.

In an example of the present aspect of the disclosure, irrigation usage may be calculated based on the irrigation pump encoder count for tracking the activation time of the irrigation pump. For example, the calculation of balanced salt solution used in irrigation is the total encoder count of the irrigation pump being in an at least partially open position for a surgical case calculated to counts/ml. In an example, one motor pump is used for irrigation. The software (e.g., in the instrument host (IH), the system controller (SC) and/or in the GUI software) starts to track the encoder count for the irrigation pump at time intervals, e.g., every 1 to 15 seconds, preferably every 10 seconds. When the encoder count is reported from a fluidics controller to the IH, SC and/or GUI, a conversion factor is used to convert the encoder count to milliliters (ml) of fluid used and subtracted from the starting fluid amount when a surgical pack is inserted into the console by a user when indicated on the GUI. The system will stop tracking the irrigation motor encoder count when the surgical pack is ejected from the console. If a bottle change occurs (e.g., during surgery), the software accounts for a new starting fluid amount and tracks from that new starting amount. If the encoder count value from the fluidics controller reaches the maximum value of the defined data type (e.g., encoder count), the software needs to track that the value has wrapped around or restarted from the beginning again. The encoder is incremental at a very fast rate and there is a possibility to wrap around, and it is dependent on size or number of bytes that store the encoder count or data type. In an example, the encoder count may range from 0 to 65535 (e.g., with a 2 byte motor), but may vary depending upon the motor used. In another example, the motor may be 4 bytes and there may wrap at a larger number (e.g., 4,294,967,295). In another example, the encoder count range may have to be traveled/traversed a few times to empty one bottle of balanced salt solution, e.g., a 500 ml bottle/bag.

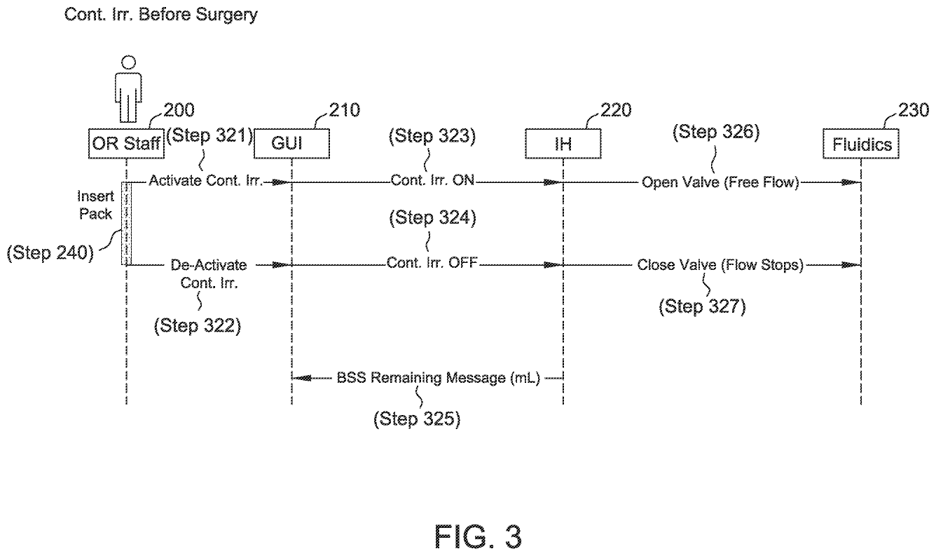

As illustrated in FIG. 3, continuous irrigation may be activated (Step 321) and can be used prior to surgery to perform a similar function as cup fill and may be activated by a user 200 of the console through the GUI 210 after insertion of the surgical pack (Step 240). The IH 220 may receive an indication to turn on continuous irrigation (Step 323) and record a start time associated with the opening of an irrigation valve (Step 326) in the fluidics assembly 230. The IH 220 may then receive an indication (Step 322) to turn off continuous irrigation (Step 324) and may record an end time associated with the closing of the previously open irrigation valve (Step 327) in the fluidics assembly 230. The IH 220 may then calculate the time elapsed between the opening and closing of the irrigation valve. The IH 220 may then multiply the elapsed time by a flow rate which is related to the balanced salt solution container height through the irrigation tubing resulting in the amount of balanced salt solution used during the use of continuous irrigation. That resulting volume may be added to any previously calculated volume of balanced salt solution usage, with the resultant subtracted by the known balanced salt solution container volume to yield the volume of remaining balanced salt solution available to the console. The IH 220 may then send an indication (Step 325) back to the GUI 210 so the user of the console knows the amount of balanced salt solution remaining available for use.

In an example of the present aspect of the disclosure, the IH 220 cumulatively tracks the calculated amounts of balanced salt solution used during each use of balanced salt solution. This may allow the IH 220 to incrementally subtract each use of balanced salt solution from the original total available to the console. The volume of balanced salt solution tracked by the IH 220 may be reset by the user when and if a second (or more) balanced salt solution container is needed to continue with a surgery and/or when the system is reset to begin a new surgical procedure. In addition, the container volume may also need to be adjusted if the second balanced salt solution container volume is not the same as the first container volume to properly track usage.

As described above, the flow rate of the balanced salt solution may be impacted by the height of the balanced salt solution container, by the pressurization of the balanced salt solution container, or the speed of the pump. In an example of the present aspect of the disclosure, the IH 220 may receive an indication of the bottle height through user input or automatically through the mechanical control by the console over the height of the balanced salt solution container. Similarly, the console may provide pressurization which may be controlled by a user of the system. For example, the balanced salt solution container may be pressurized to provide a flow rate of about 120 ml/min to about 160 ml/min. In addition, pump variables including, but not limited to, speed, steps and/or encoder counts or irrigation rate may be communicated to the IH.

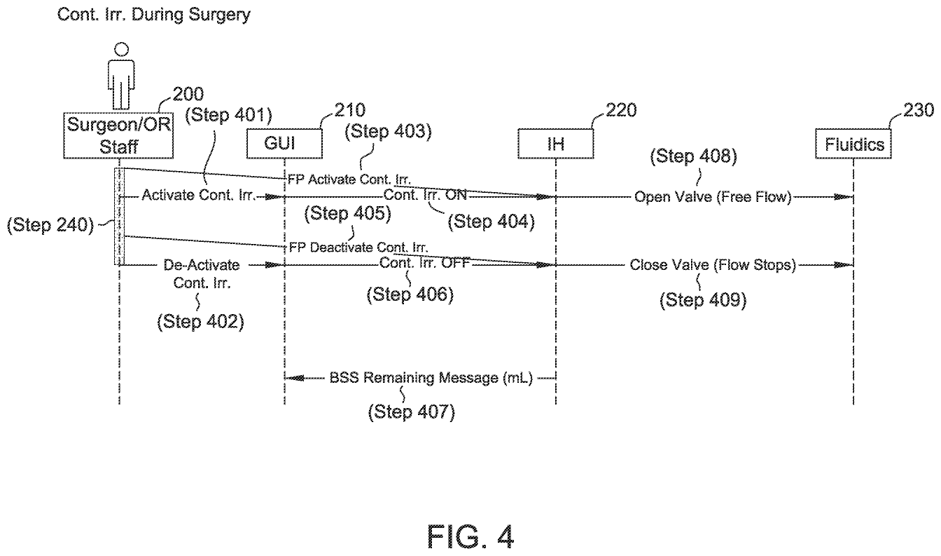

Continuous irrigation may continue during surgery as illustrated in FIG. 4 and may be additionally controlled using an input device such as, for example, a foot pedal controller. The foot pedal may communicate directly with the IH 220 which may calculate the balanced salt solution usage during continuous irrigation in the same way as discussed above, e.g., by tracking the time between opening and closing an irrigation valve, the flow rate according to either balanced salt solution container height, pressurized container and/or pump variables in accordance with commands received from the foot pedal.

As illustrated in FIG. 4, continuous irrigation may be activated (Step 401) and the IH 220 may receive an indication to turn on continuous irrigation (Step 404), through the use of the foot pedal (Step 403) and record a start time associated with the opening of an irrigation valve (Step 408) in the fluidics assembly 230. The IH 220 may then receive an indication (Step 402) to turn off continuous irrigation (Step 406), through the use of the foot pedal (Step 405), and may record an end time associated with the closing of the previously open irrigation valve (Step 409) in the fluidics assembly 230. The IH 220 may then calculate the time elapsed between the opening and closing of the irrigation valve. The IH 220 may then multiply the elapsed time by a flow rate which is related to the balanced salt solution container height through the irrigation tubing resulting in the amount of balanced salt solution used during the use of continuous irrigation. That resulting volume may be added to any previously calculated volume of balanced salt solution usage, with the resultant subtracted by the known balanced salt solution container volume to yield the volume of remaining balanced salt solution available to the console. The IH 220 may then send an indication (Step 407) back to the GUI 210 so the user of the console knows the amount of balanced salt solution remaining available for use.

In an example of the present aspect of the disclosure, the IH 220 may further take into account wound leakage during a surgical procedure and may add a wound leakage value to the total calculated balanced salt solution usage between each open and closed irrigation valve cycle. Wound leakage may be impacted by needle gauge, sleeve size, and/or incision size, including from any second incision point. In an example of the present aspect of the disclosure, a prepopulated data table may be available to the IH 220 and may include various wound leakage volumes sorted by time, needle size, and sleeve size. Although values for needle size and sleeve size may be entered by the user into the GUI 210, during a prime sequence of the console, the tip size and/or sleeve size may be calculated.

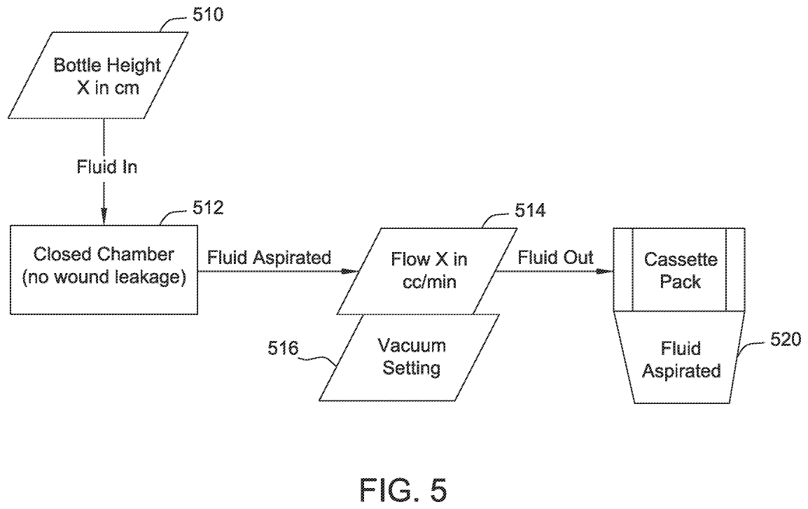

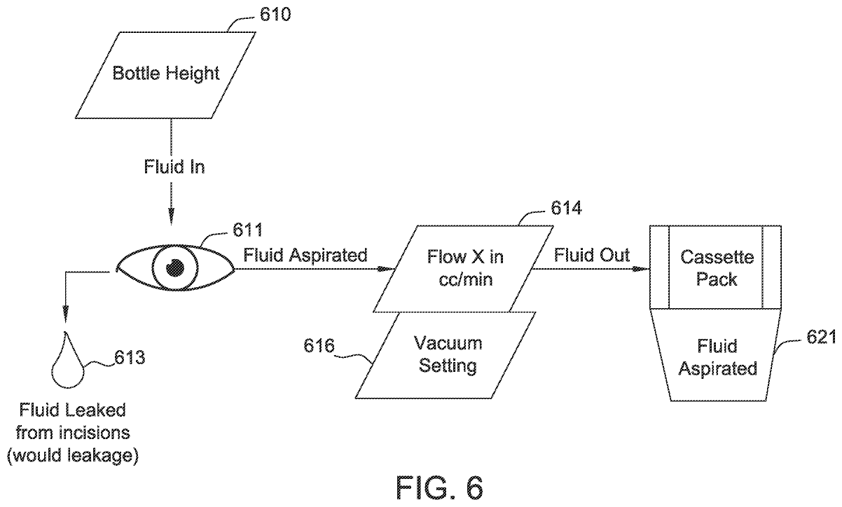

In another example, as illustrated in FIG. 5, wound leakage can be calculated by first calculating a theoretical total amount of fluid used 520 in a closed chamber 512 based on flow 514, container height 510, and vacuum settings 516. In this example, wound leakage is not a factor. In another example, during surgery, as illustrated in FIG. 6, wound leakage is a factor and the aspirated volume 621 tracked by the drain encoder count at similar flow 614, container height 610 and vacuum settings 616, can be subtracted from the theoretical total amount of fluid used 620 to estimate the amount lost due to wound leakage 613. This calculated wound leakage can still vary due to many factors, including, but not limited to, eye 611 type, wound stretching, surgical technique, and a would leakage factor can be entered in the GUI 210 to adjust for higher or lower than calculated amount of leakage.

In an example of an aspect of the disclosure, assuming a surgical procedure without any leakage (a “Perfect Condition”), then amount of balanced salt solution received by the drain bag may be estimated based on flow rate setting, time duration of the foot pedal in zone 1/2/3, tip size, sleeve size, and other factors. However, in a typical surgery, some balanced salt solution may leak from the surgical site and would not be collected by the drain bag. This is generally referred to as wound leakage. In an example of an aspect of the disclosure, wound leakage can be estimated by subtracting from the Perfect Condition (estimated thru different factors) the amount of balanced salt solution actually collected in the drain bag.

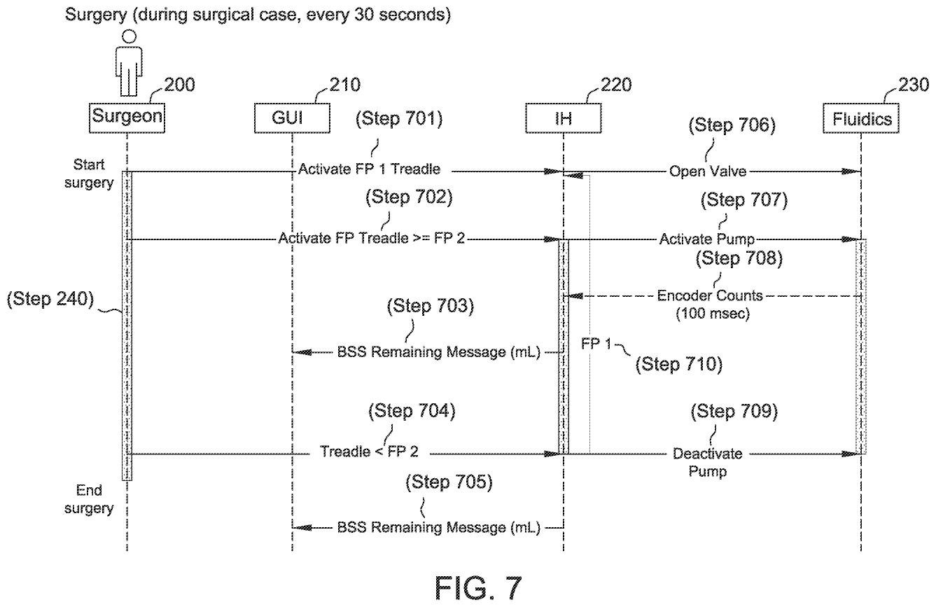

During the portion of a surgical procedure where aspiration and irrigation are more continuously used, the flow of fluid away from the surgical site may be the best indicator of total fluid usage. As illustrated in FIG. 7, the amount of balanced salt solution used during surgery may also be calculated during a continuous surgical period and may include calculations based on the amount of fluid aspirated from the surgical site. In an example of the present aspect of the disclosure, a surgeon may use a foot pedal (at position FP 1) to activate (Step 701) and open the irrigation valve (Step 706) for continuous irrigation, allowing balanced salt solution fluid to flow to the surgical site. Moving the foot pedal to the next activation positions (FP 2 or FP 3) (Step 702) activates an aspiration pump (Step 707) facilitating the removal of fluid from the surgical site through an aspiration line into a drainage bag associated with the surgical cassette. During this stage of surgery while balanced salt solution is being both delivered to and removed from the surgical site, the present aspect of the disclosure may calculate and provide to the user an updated indication of how much balanced salt solution remains available (Step 703) to the console.

In an example of the present aspect of the disclosure, once the aspiration pump has been activated in the irrigation/aspiration (FA) phase of surgery, a calculation based on the amount of time the aspiration pump is active is completed in predetermined time intervals to provide the user of the console real-time indications on the amount of balanced salt solution available to the console. More specifically, the IH 220 may track and accumulate from an encoder counts measured in time, for example, 100 millisecond increments, to indicate the length of time the aspiration pump is active. The IH 220 may use cumulated counts, handpiece tip size information, and known aspiration rates to calculate a volume of balanced salt solution aspirated by the pump. This value is added to any previous balanced salt solution usage volumes calculated by the IH, a wound leakage volume as described above, and the amount of balanced salt solution dispensed to the surgical site prior to the activation of the aspiration pump (which may be calculated using the same calculations used for continuous irrigation as described above). This calculated value may be provided to the GUI 210 every 30-40 milliseconds (Step 708), for example, during active irrigation and aspiration to update the user of the system. Once the aspiration pump is deactivated (Step 709) after the IH 220 received an indication from the foot pedal (Step 704), any additional balanced salt solution usage may be calculated using the same calculations used for continuous irrigation as described above, with an indication of the balanced salt solution usage returned to the GUI 210 (Step 705). Similarly, irrigation usage may be calculated based on the irrigation pump encoder count as described above.

The prime/tune function of a phacoemulsification console must occur prior to each procedure, anytime a handpiece is reattached to the console, and/or after the surgical cassette is inserted or replaced. The prime/tune process may fill the I/A tubing with fluid, perform a system vacuum check, and test and characterize the handpiece. The prime/tune sequence may be partly bypassed by using continuous irrigation to fill the irrigation tubing of the surgical cassette, leaving only a tune process to be completed. During a full prime/tune sequence, for example, the present aspect of the disclosure calculates the fluid used during system prime and tune operation using encoder data from the drain pump. The fluid in the irrigation line and the surgical cassette gets evacuated with the peristaltic drain pump to the waste bag, which is generally associated with the surgical cassette.

In an example of the present aspect of the disclosure, in the absence of a drain pump, for example, a predetermined amount of fluid may be used to fill the tubing of a cassette and would be used as a constant in the calculation to determine the amount of remaining balanced salt solution.

The present aspect of the disclosure may account for fluid volume of the irrigation, aspiration tubing and cassette during the prime/tune process. In an alternate example, the encoder data can be used from the irrigation pump to derive the amount dispensed during prime/tune sequence and subtract the volume retained in the cassette and tubing to calculate the fluid used.

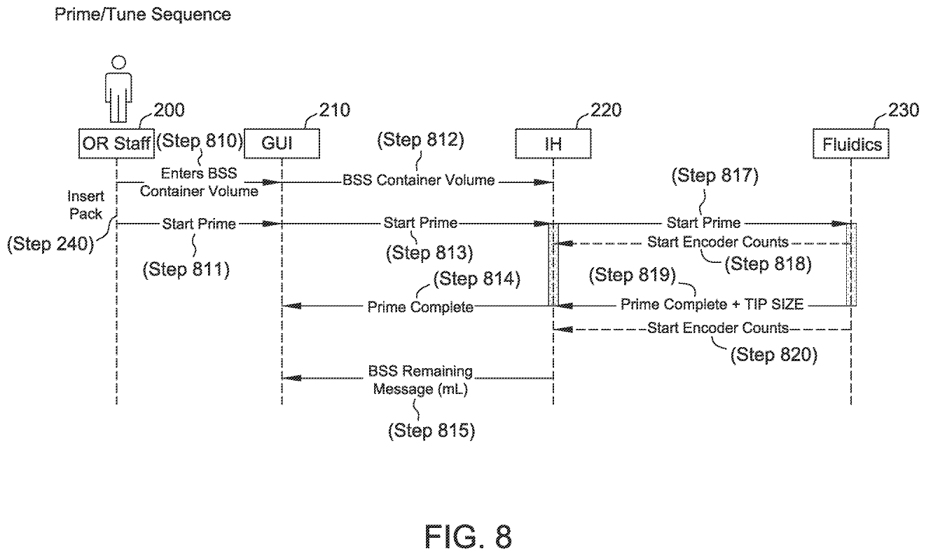

As illustrated in FIG. 8, the prime/tune sequence may begin, after a surgical pack is inserted into the surgical console at Step 240, with a user 200 entering into the GUI 210 (Step 810) the volume of balanced salt solution available to the surgical console which may then be recorded to the IH 220 (Step 812) which is in communication with various other components of the surgical system which may use the available volume of balanced salt solution. Additionally, the surgical console may carryover the unused balanced salt solution from the previous procedure. For example, if the user started a first procedure with a new 500 ml balanced salt solution bottle, and used 225 ml during the first procedure, at the start of the next procedure, the GUI may indicate that 275 ml is available for use in the current (e.g., second) procedure.

A user 200 may provide an indication to begin the prime sequence (Step 811) which activates the prime cycle (Step 816) through the GUI 210 (Step 813) which prompts the IH 220 to open the irrigation valve and active the aspiration pump, tracking with encoder (Step 818), to fill the I/A tubing with fluid and perform a vacuum check. The IH 220 may utilize encoder counts to track (Step 818) and calculate the amount of fluid used by the aspiration pump of the fluidics assembly 230 during the prime sequence which may be automatically concluded by the console (Step 819) with an indication of whether the prime sequence was successful sent from the IH 220 to the GUI 201 (Step 814). In addition, the calculated amount of fluid used by the aspiration pump may be added to the amount of fluid necessary to fill the I/A tubing as well as any measurable amount delivered to the aspiration tank.

Using the indication received from the IH 220, the GUI 210 may alert the user to the status of the amount of balanced salt solution remaining/available to the console (Step 815). The GUI 210 may, for example, alert the user in varied ways depending on the amount of balanced salt solution remaining. For example, the GUI 210 may display the estimated amount left, a percentage amount left in the original balanced salt solution container and/or may change the color of the indication based on the amount left or when the percentage left falls below a predetermined threshold, such as 30% followed by reminders after additional 10% is used. The predetermine threshold may also be an absolute value, such as 100 ml, 150 ml, or 200 ml. Similarly, the system may provide an audible or haptic alert when a predetermined threshold or other desired amount or point in time is reached. As would be understood by those skilled in the art, the type of alerts can take any number of forms.

To best ensure a timely indication to the user, the system may refresh the calculations of balanced salt solution usage at a predetermined interval, for example, every 100 milliseconds, to ensure that the user of the console is aware of the amount of balanced salt solution remaining for use. Any predetermined interval may be used based on one or more system parameters and/or desired or required information for proper function of the system. The system may, for example, pause the surgical functionality of the system when the amount of balanced salt solution remaining falls below a predetermined threshold, such as 10% and/or 50 ml remaining, for example. Such an action may greatly increase the likelihood that the user would switch to a new balanced salt solution container prior to complete depletion, thereby resetting the amount of BBS fluid available for use. Similarly, the user may be given the option to bypass the pause in surgical functionality knowing that what balanced salt solution remains is sufficient to finish the surgical procedure or simply chooses to change the balanced salt solution source at a later time. The alerts provided by the system are designed to warn and prevent the user from unknowingly running out of balanced salt solution which can lead to surgical complications and prime/tune issues with the system.

In addition to the algorithm defined above, the algorithm can add a sensor that provides additional inputs in place of certain parts of the algorithm to determine the balanced salt solution usage. The sensor may be, but is not limit to, a weight sensor, a photosensor, or an in-line flow sensor.

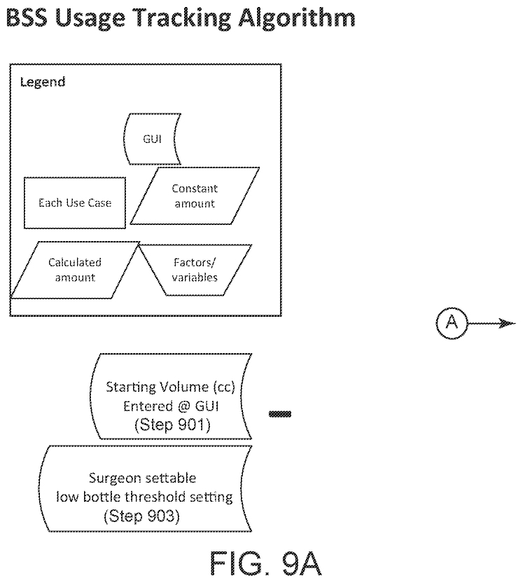

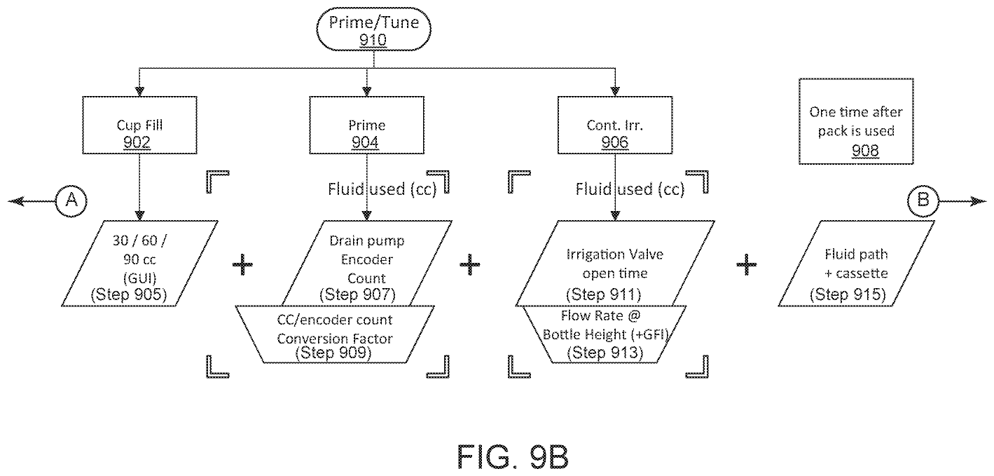

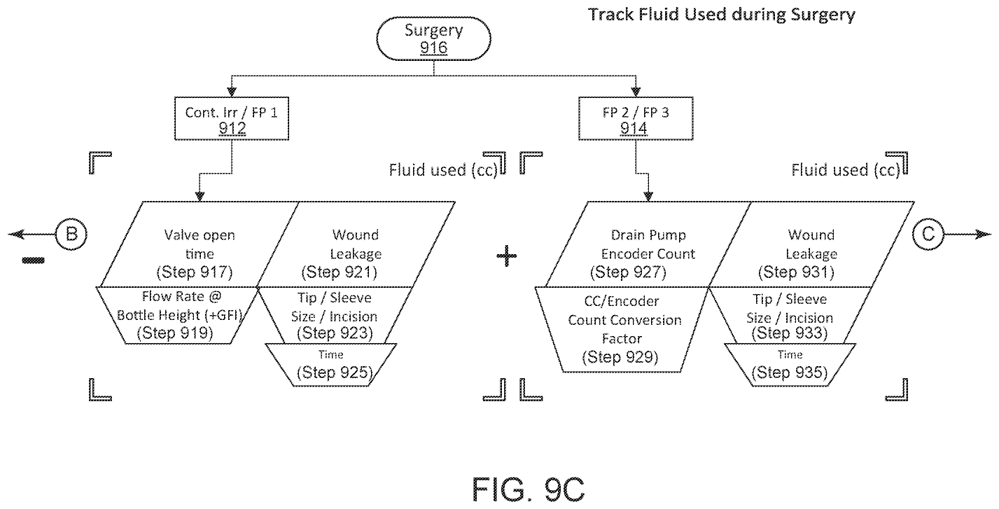

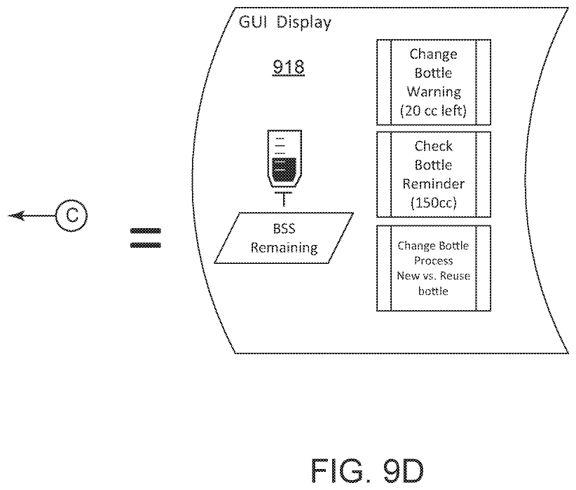

A flowchart illustrating an exemplary algorithm of the present aspect of the disclosure is illustrated in FIGS. 9A-9D. A legend for FIGS. 9A-9D is provided in FIG. 9A for clarification. As illustrated in FIG. 9A, the starting volume of balanced salt solution may be received by the GUI 210 (Step 901) by manually entering the volume and/or a barcode or QR code may be scanned by the system, for example. Similarly, other variables may be inputted into the GUI 210, such as, for example, thresholds for activating a system warning related to low balanced salt solution availability (Step 903).

As illustrated in FIG. 9B, as described above, various system functions which may use a volume of balanced salt solution may be tracked as to their balanced salt solution usage which may be subtracted from the starting volume of balanced salt solution entered into the GUI 210. For example, during the Prime/Tune Sequence 910, each of the possible functions of Cup Fill 902, Prime 904, and Continuous Irrigation 906, may impact the starting volume of the balance salt solution. For example, if Cup Fill 902 is engaged, a volume of balance salt solution may be dispensed (Step 905). During Prime 904, the use of balance salt solution may be calculated by applying a conversion factor (step 909) to a drain pump encoder count (Step 907). Similarly, for example, the use of Continuous Irrigation 906 may track the usage of balance salt solution by tracking the irrigation valve open time (Step 911) and the flow rate of balance salt solution from the source of the balance salt solution (Step 913). Each of the three functions illustrated may or may not necessarily occur during each pre-surgical procedure and each function may occur at any time during the use of the console. In addition, if a cassette has had prior use, the amount balance salt solution may be calculated by function 908 based on the known capacity of the cassette (Step 915).

An additional volume of balanced salt solution usage may be tracked during surgery as illustrated in FIG. 9C. As more fully described above, various foot pedal positions may control various aspects of console functions during active surgery and may use balanced salt solution which usage may be tracked and additively subtracted from the amount of balanced salt solution available to the console. For example, a first foot petal position 912 may provide for continuous irrigation and the balance salt solution usage may be calculated from the measured valve open time (Step 917) and the balance salt solution flow rate (Step 919). Additional factors correspondent to the use of the balance salt solution during Surgery 916 may include an estimate of wound leakage (Step 912), the known handpiece tip and/or sleeve size and estimate incision size (Step 923), each over the run time of a first foot petal position 912 (Step 925). As second/third foot pedal position (FP2 and/or FP3) 914 may provide for the delivery and removal of balanced salt solution to and from the surgical site which may be calculated using a conversion factor (Step 929) against a drain pump encoder count (Step 927). The calculated usage of balanced salt solution at second/third foot pedal position 914 may include an estimate of wound leakage (Step 931), the known handpiece tip and and/or sleeve size and estimate incision size (Step 933), each over the run time of second/third foot pedal position 914 (Step 935) (i.e., amount of time in FP2 and/or FP3). As an example, the conversion factor at Step 929 may be equal to a drain pump encoder count of 2550, which may be equal to one milliliter. As may be appreciated by those skilled in the art, console functions may vary between different types and brands of surgical consoles and may be controlled or divided in different ways. Thus, for example, the surgical functions as illustrated in FIG. 9C may be combined into a single function and/or may include additional functionalities which may impact the amount of balanced salt solution available to the console.

In an example of the present aspect of the disclosure, the amounts of balanced salt solution tracked over FIGS. 9B and 9C may be added together and subtracted by the amount provided in FIG. 9A to provide a value of remaining balanced salt solution in GUI Display 918 as illustrated in FIG. 9D. The GUI may, as described herein, present various warnings related to the amount of available balanced salt solution and to how surgical functions may be impacted. The GUI may also provide menu access to various system functions, such as those to change or alter the amount of balanced salt solution available to the console after an initial starting of the system.

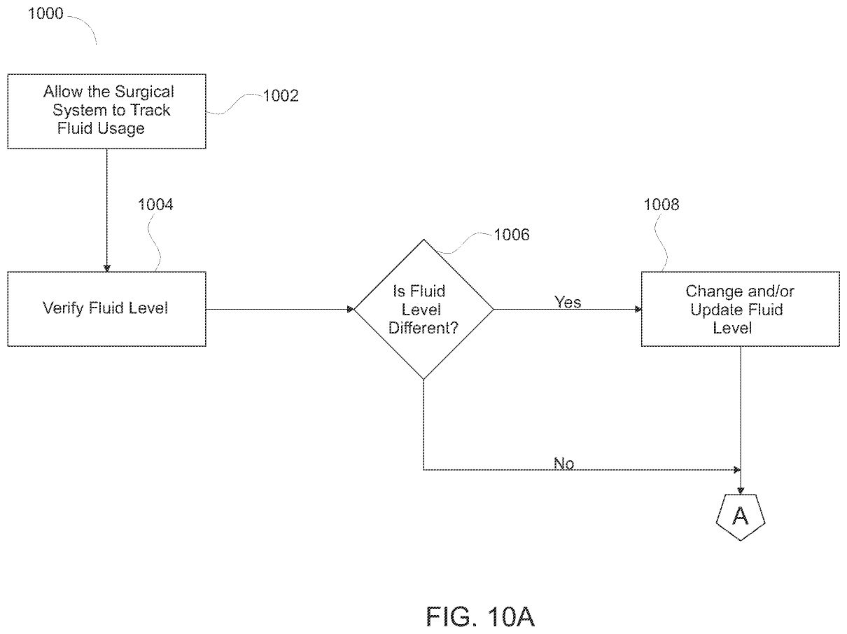

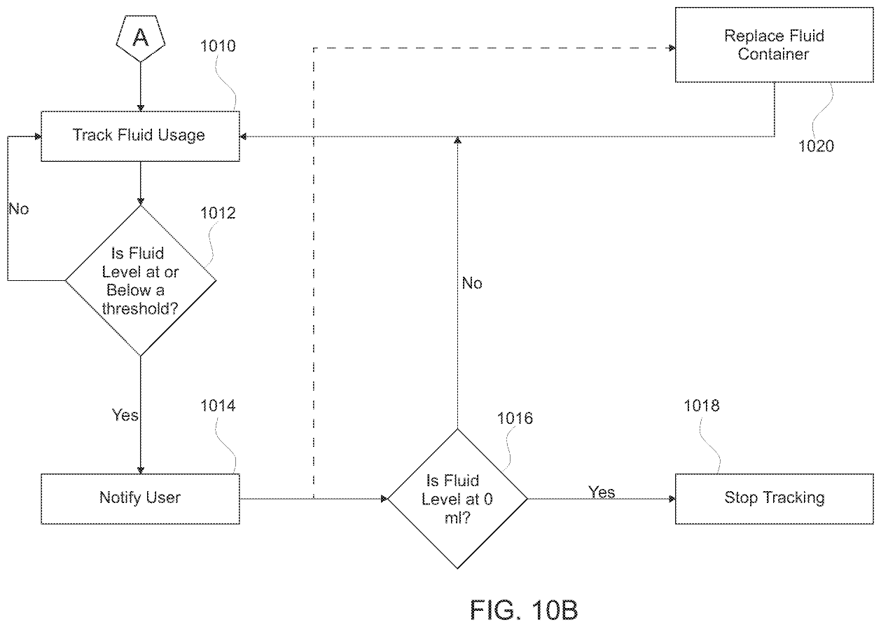

FIGS. 10A and 10B illustrates an exemplary algorithm for a warning system designed to alert the user when the balanced salt solution (e.g., fluid) is below a preset threshold value. The surgical console may include a processor configured to perform the algorithm disclosed in FIGS. 10A and 10B.

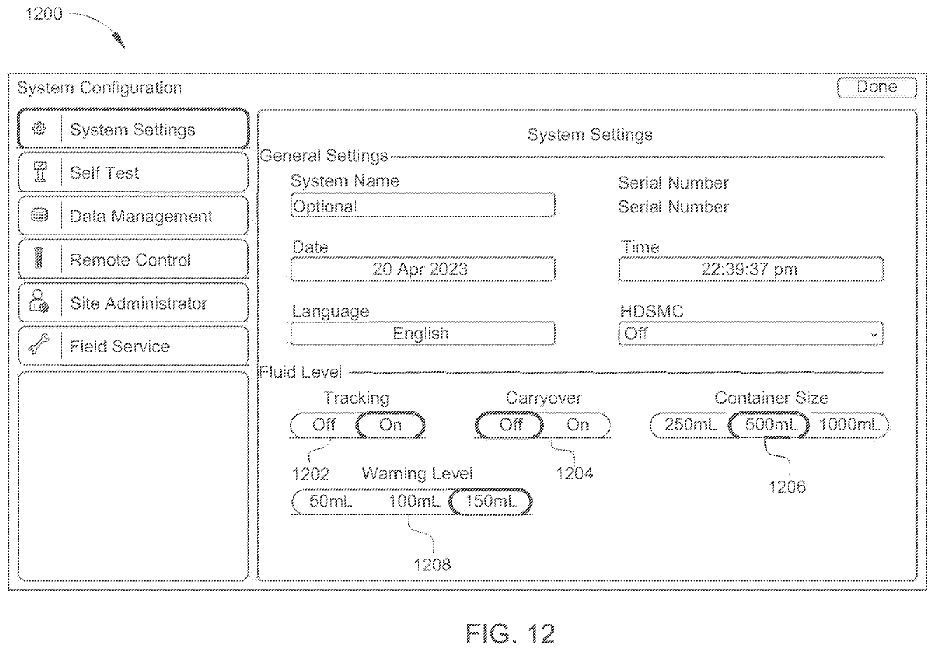

Starting with FIG. 10A, at 1002, the user allows the surgical system to track the usage of the balanced salt solution. The tracking may be set up via the GUI shown in FIG. 12.

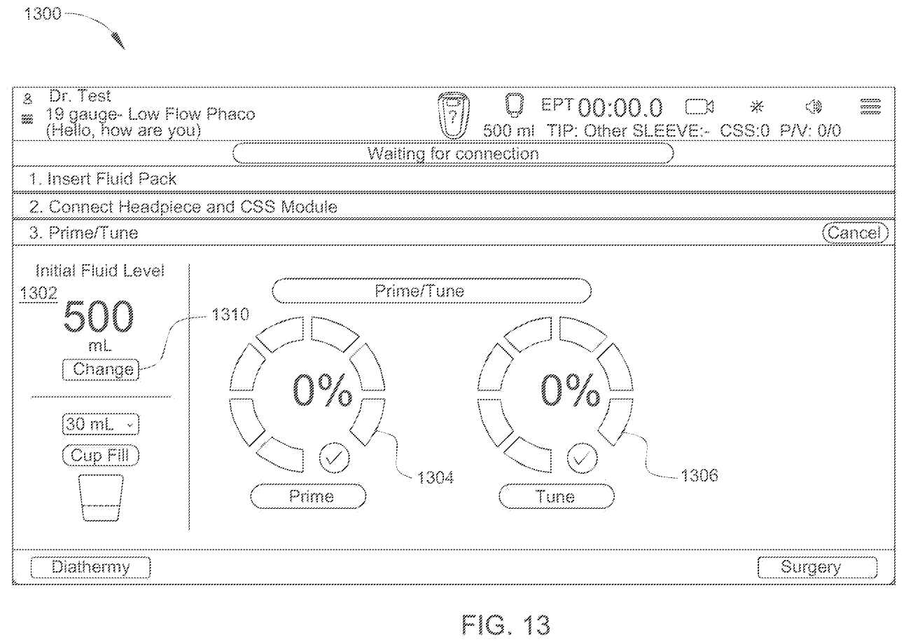

At 1004, via, for example, the GUI shown in FIG. 13, the user may verify the starting balanced salt solution amount or level.

At 1006, the user/surgical system checks whether the amount of balanced salt solution in the container is different.

At 1008, if the balanced salt solution amount or level is different, the user, for example, via the GUI shown in FIG. 13, may change and/or update the starting balanced salt solution amount or level.

If the balanced salt solution is not different at step 1006, then moving to FIG. 10B, at 1010, the surgical system tracks the balanced salt solution usage and levels as described herein.

At 1012, the surgical system may check whether the balanced salt solution is at or below a preset threshold (e.g., 25 ml, 50 ml, or 100 ml).

At 1014, if the balanced salt solution is at or below the preset threshold the surgical system may notify the user that the fluid is low. The notification may be a text warning via the surgical console's GUI. The notification may also be an audio notification warning the user that the level has fallen below the threshold. The notification may also be the graphical text of the fluid level indicator on the GUI changing colors (e.g., yellow font to red font). If the balanced salt solution is not at or below the preset threshold at step 1010, the surgical system may continue to track the balanced salt solution level as in step 1010. If a user carries over a balanced salt solution container from a previous procedure, and the container is below the predefined threshold, the surgical console's GUI will display a warning to the user that the balanced salt solution is low.

At 1020, the user, if they see the warning, may replace the balanced salt solution container. After the balanced salt solution container has been replaced, the surgical system may begin to track usage at step 1010.

At 1016 and if the fluid level is zero, the surgical system will stop tracking the balanced salt solution usage at step 1018. Furthermore, or alternatively, the surgical system may pause, shutdown, or go into a safe mode.

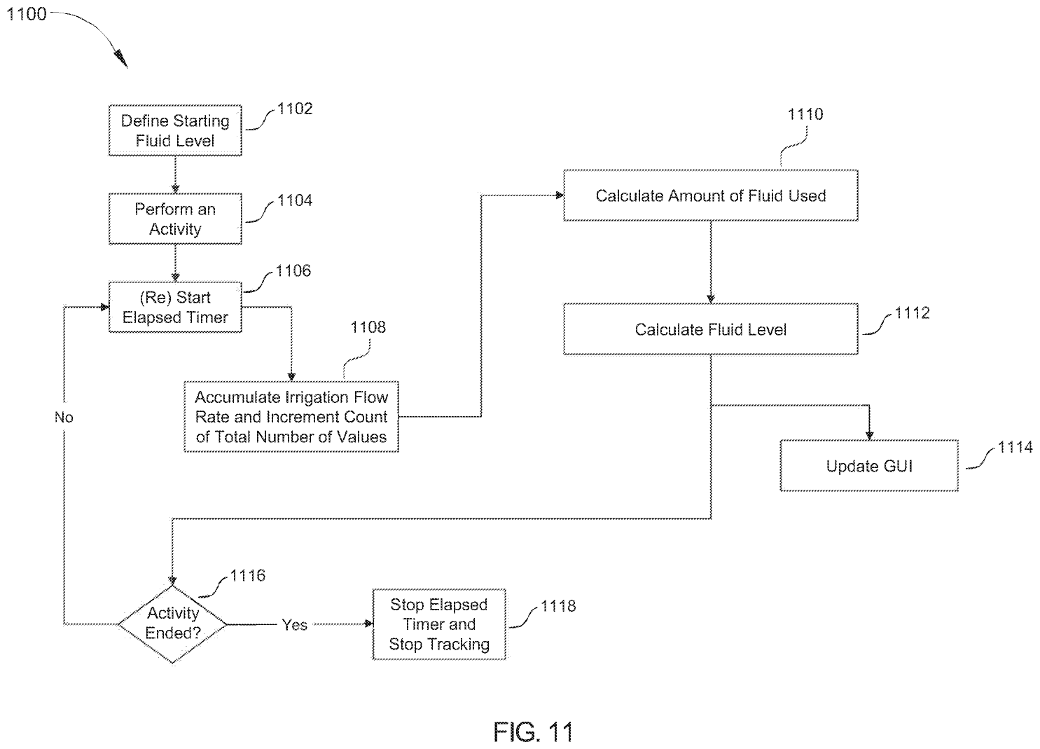

A flowchart illustrating an exemplary algorithm 1100 of a present aspect of the disclosure is illustrated in FIG. 11. At step 1102, the user may define the initial balanced salt solution amount or level. In one example, the user may select from a pre-populated list of bottle sizes, such as, 250 ml, 500 ml, and 1000 ml.

In another example, the initial balanced salt solution level may be a carryover from the previous procedure. In this instance, the user may not need to input the starting level because the system may carryover the leftover amount from the previous procedure. For example, if the user started a first procedure with a new 500 ml balanced salt solution bottle, and used 225 ml during the first procedure, at the start of the next procedure, the initial balanced salt solution may be 275 ml.

At 1104, the user may perform an activity, such as a cup fill, prime procedure, reflux, or a surgical procedure. At 1106, the system starts an elapsed timer. In one example, the elapsed timer may update every 10 seconds.

At 1108, the system measures the encoder count of the irrigation motor and accumulates the irrigation flow rate, including positive counting, negative counting, and zero counting. The system may measure the encoder count of the irrigation motor and accumulates the irrigation flow rate every 15 to 20 ms.

Negative counting must be accounted for because in some surgical systems, the irrigation pump may spin in a reverse direction. The irrigation pump may include an encoder to track the rotation of the pump. The irrigation pump may rotate in a reverse direction to manage intraoperative and/or intraocular pressure in the ocular chamber when the chamber exceeds a target pressure by a specified amount. The irrigation pump may also rotate in a reverse direction to maintain ocular chamber pressure during a procedure. When the irrigation pump runs in a reverse direction, the balanced salt solution may go from the irrigation line back into the cassette and subsequently back towards or into the irrigation inlet and drip chamber.

In an optional example, negative counting may also account for operation of the aspiration pump. in instances where the aspiration pumps may spin in a reverse direction. Similar to the irrigation pump, the aspiration pump may include an encoder to track the rotation of the pump. For example, the aspiration pump may rotate in a reverse direction to apply positive pressure to the ocular chamber for venting and/or reflux to maintain the intraocular pressure at a desired level. Accordingly, to get an accurate measurement of the amount of balanced salt solution used during the surgical procedure, the algorithm may consider backward rotation of the aspiration pump and subtract the aggregated fluid volume from the current balanced salt solution value because balanced salt solution may have gone back into the irrigation inlet and drip chamber.

Further at 1110, the encoder count is converted to an irrigation flow rate (cc/min or ml/min). As long as the user's activity continues, the encoder will continue to accumulate the irrigation flow. The surgical system will calculate the amount of balanced salt solution used. In one example, the system may use Equation 1 below to calculate the amount of solution used:

$$Solution Used = ( ( \frac{Accumulated Irrigation Flow}{\begin{matrix}Count of Total Number \\ of Accumulated Values\end{matrix}} ) \times Elapsed Time ) \div 60$$

The accumulated irrigation flow is the total measured irrigation while the counter was running. The count of the total number of accumulated value is the total number of measurements that were taken. The accumulated irrigation flow divided by the Count of Total Number of Accumulated Values is the irrigation rate. The elapsed time is the total time the counter was running.

If, at any time during the algorithm described in FIG. 11, an error occurs causing an interruption in the data streamed from the encoder to the surgical console, the surgical console will pause the tracking and calculate the solution used up to the point of the error. When the error is resolved, the surgical console will start tracking balanced salt solution usage.

At 1112, the new balanced salt solution amount or level is calculated by subtracting the balanced salt solution used from the previous fluid amount or level. At 1114, the GUI is updated to display the new fluid amount or level.

After the system calculates the fluid level at 1112, it determines whether the activity ended at 1116. If the activity has ended, at 1118, the system stops the elapsed timer and stops tracking fluid usage. If the activity has not ended, the system goes back to 1106 where the elapsed time is restarted and the system continues to accumulate the irrigation flow rate at 1108 and perform the subsequent steps.

FIG. 12 shows an example GUI 1200 for the system settings. The GUI 1200 may include a Tracking setting 1202, Carryover setting 1204, Container Size setting 1206, and Warning Level setting 1208. The Tracking setting 1202 allows the user to turn on the balanced salt solution usage tracking feature. The Carryover setting 1204 allows the user to carryover the fluid amount or level from a previous procedure. The Container Size setting 1206 allows a user to indicate the size of the balanced salt solution bag or bottle being used. The Warning Level setting 1208 allows a user to set at what remaining amount of balanced salt solution the system will provide a warning to the user.

FIG. 13 is an example GUI 1300 showing the Prime/Tune part of the system set up procedure. The GUI 1300 includes an Initial Fluid Level 1302, Prime progress 1304 and Tune progress 1306. Via a change toggle 1310, a user may adjust the initial fluid level the user is starting with for the procedure. The initial fluid level may be the size of the balanced salt solution being used (e.g., 250 ml, 500 ml, or 1000 ml). The initial fluid level may also be the amount of balanced salt solution that was carried over from a previous procedure.

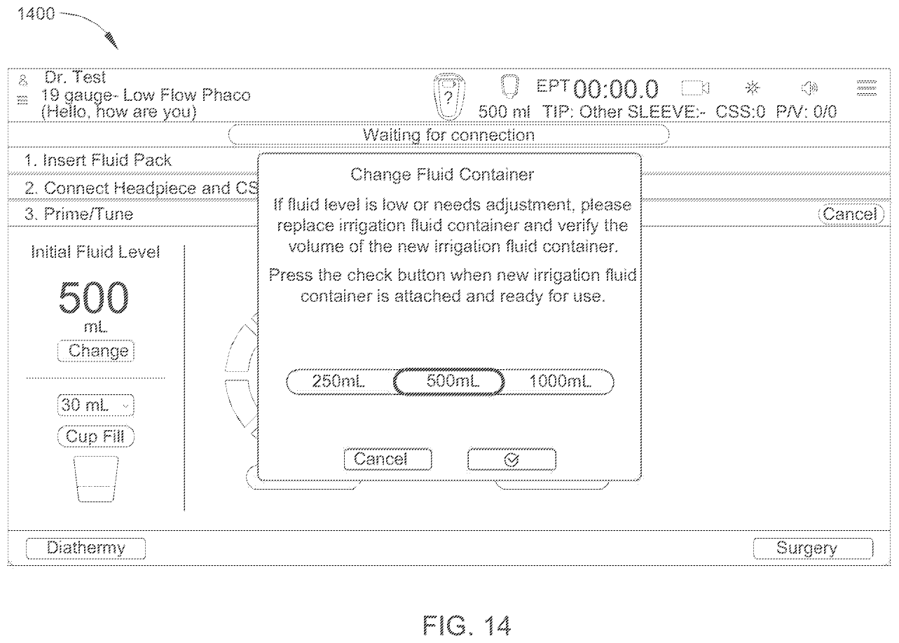

FIG. 14 shows an example GUI 1400 for when the user selects the Change toggle 1310 in FIG. 13. As shown in FIG. 14, the user may select from three different default balanced salt solution sizes—250 ml, 500 ml, and 1000 ml.

### EXAMPLES

### Example 1

A system for monitoring the amount of fluid used during an ophthalmic surgical system comprising: a fluid source connected to a surgical console having a known volume of fluid, a cassette having a known fluid capacity, an encoder for tracking at least one pump, and an irrigation valve control providing a plurality of state indications, wherein the amount of fluid removed from the fluid source is calculated by summing the plurality of state indications and the fluid capacity of the cassette.

### Example 2

The system of example 1, wherein each state indication is indicative of an open or closed valve position.

### Example 3

The system of any one of examples 1 to 2, wherein the encoder tracks activity of a drainage pump that is in communication with the cassette.

### Example 4

The system of any of the examples 1 to 3 wherein the amount of fluid removed from the fluid source in calculated by further summing the amount of fluid associated with the encoder tracked activity of the drainage pump.

### Example 5

The system of any of the examples 1 to 4, wherein the encoder tracks activity of an irrigation pump that is in communication with the cassette.

### Example 6

The system of any of the examples 1 to 5, wherein the amount of fluid removed from the fluid source is calculated by further summing an amount of fluid associated with wound leakage.

### Example 7

The system of any of the examples 1 to 6, wherein the fluid capacity of the cassette includes a handpiece volume and an aspiration and irrigation tubing.

### Example 8

The system of any of the examples 1 to 7, wherein the known volume of fluid is an initial size of the fluid source coupled with the surgical console.

### Example 9

The system of any of the examples 1 to 8, wherein the known volume of fluid is carried over from a previous ophthalmic surgical procedure.

### Example 10

The system of any of the examples 1 to 9, further comprising a processor configured to provide a warning when an amount of fluid remaining in the fluid source drops below a threshold value.

### Example 11

A method for monitoring the amount of fluid used or remaining in an ophthalmic surgical system, comprising: receiving a supply value for the volume of fluid in a fluid source available to a surgical console, calculating, by an instrument host of a surgical console, the amount of fluid remaining in the fluid source, wherein the instrument host receives values for one or more of the following: the fluid capacity of a surgical cassette, the leakage of fluid from a surgical site, the measuring of drainage activity by the drainage pump encoder, the plurality of state indications for an irrigation valve control, the measuring of aspiration activity by the aspiration pump encoder, the height of the fluid source available to the surgical console, the vacuum setting of the surgical console, or the aspiration flow rate of the surgical console, wherein the amount of fluid remaining in the fluid source by the surgical console is calculated by subtracting from the received supply value of the fluid in a fluid source the sum of the amount of fluid associated with each of the instrument host received values, and sending to a display of the console, an indication of the calculated amount of fluid usage.

### Example 12

The method of example 11, wherein each state indication is indicative of an open or closed valve position.

### Example 13

The method of any of the examples 11 to 12, wherein the calculating occurs according to a predetermined time threshold.

### Example 14

The method of any of the examples 11 to 13, wherein the calculating occurs at least every 30 milliseconds.

### Example 15

The method of any of the examples 11 to 14, wherein the drainage pump encoder is measured once at least every 100 milliseconds.

### Example 16

The method of any of the examples 11 to 15, wherein the activity of the drainage pump is selected from the group consisting of pump runtime, pump speed, and direction.

### Example 17

The method of any of the examples 11 to 16, wherein the instrument host calculates a value for wound leakage.

### Example 18

The method of any of the examples 11 to 17, wherein the fluid capacity of the cassette includes a handpiece volume.

### Example 19

The method of any of the examples 11 to 18, wherein the fluid capacity of the cassette includes a volume of an irrigation and aspiration tubing.

### Example 20

The method of any of the examples 11 to 19, wherein the supply value is an initial size of the fluid source coupled with the surgical console.

### Example 21

The method of any of the examples 11 to 19, wherein the supply value is carried over from a previous ophthalmic surgical procedure.

### Example 22

The method of any of the examples 1 to 21, further comprising providing a warning when an amount of fluid remaining in the fluid source has drops below a threshold value.

### Example 23

A system for monitoring the amount of fluid used during an ophthalmic surgical system, comprising: a fluid source connected to a surgical console having a known volume of fluid, a cassette having a known fluid capacity, and an input device for receiving the amount of fluid requested for a cup fill, and an irrigation valve control tracking the open time of the irrigation valve, wherein the flow rate of the fluid from the fluid source and the height of the fluid source is factored with the irrigation valve open time to provide an indication wherein the amount of fluid removed from the fluid source is calculated by summing the amount of fluid associated with the plurality of state indications and the fluid capacity of the cassette.

### Example 24

The system of any one of example 23, wherein the amount of fluid removed from the fluid source is calculated by further summing an amount of fluid associated with wound leakage.

### Example 25

The system of any one of examples 23 to 24, wherein the fluid capacity of the cassette includes a handpiece volume and an irrigation and aspiration tubing volume.

### Example 26

The system of any one of examples 23 to 25, wherein the known fluid capacity is an initial size of the fluid source coupled with the surgical console.

### Example 27

The system of any one of examples 23 to 25, wherein the known fluid capacity is carried over from a previous ophthalmic surgical procedure.

### Example 28

The system of any one of examples 23 to 27, further comprising a processor configured to provide a warning when an amount of fluid remaining in the fluid source drops below a threshold value.

### Example 29

In an example of the present aspect of the disclosure, a system for monitoring the amount of fluid used during an ophthalmic surgical system, comprising: surgical procedure, comprising: a fluid source coupled with a surgical console, an irrigation pump fluidly coupled with the fluid source and in communication with an encoder for tracking the activation time of the irrigation pump, and an instrument host for receiving and applying a conversion factor to the activation time for calculating the amount of fluid used during the ophthalmic surgical procedure or an amount of fluid remaining in the fluid source.

### Example 30

The system of example 29, wherein the amount of fluid removed from the fluid used during the ophthalmic surgical procedure is calculated by further summing an amount of fluid associated with wound leakage.

### Example 31

The system of any one of examples 29 to 30 further comprising a cassette having a known fluid capacity.

### Example 32

The system of example 31, wherein the fluid capacity of the cassette includes a handpiece volume.

### Example 33

The system of example 31, wherein the fluid capacity of the cassette includes a volume of an irrigation and aspiration tubing.

### Example 34

The system of any one of examples 29 to 33, wherein the conversion factor is a numeric value divisible into an encoder count to yield a volumetric value.

### Example 35

The system of any one of examples 29 to 33, wherein the conversion factor is 2550.

### Example 36

The system of any one of examples 29 to 35, wherein an amount of fluid in the fluid source is an initial size of the fluid source couple with the surgical console.

### Example 37

The system of any one of examples 29 to 36, wherein an amount of fluid in the fluid source is carried over from a previous ophthalmic surgical procedure.

### Example 38

The system of any one of examples 29 to 37, further comprising a processor configured to provide a warning when an amount of fluid remaining in the fluid source drops below a threshold value.

### Example 39

A system for monitoring the amount of fluid used during an ophthalmic surgical procedure, comprising a fluid source coupled with a surgical console, an irrigation pump fluidly coupled with the fluid source and in communication with an encoder for tracking the activation time of the irrigation pump, and an instrument host for receiving and applying a conversion factor to the activation time for calculating the amount of fluid used during the ophthalmic surgical procedure or an amount of fluid remaining in the fluid source.

### Example 40

The system of example 39, wherein the amount of fluid removed from the fluid used during the ophthalmic surgical procedure is calculated by further summing an amount of fluid associated with wound leakage.

### Example 41

The system of any one of examples 39 to 40, wherein the value of a third volume of fluid is added to the first volume of fluid.

### Example 42

The system of any one of examples 39 to 41, wherein the first volume of fluid is an initial size of the fluid source coupled with the surgical console.

### Example 43

The system of any one of examples 39 to 41, wherein the first volume of fluid is carried over from a previous ophthalmic surgical procedure.

### Example 44

The system of any one of examples 39 to 44, further comprising a processor configured to provide a warning when an amount of fluid remaining in the fluid source drops below a threshold value.

### Example 45

A method for monitoring the amount of fluid used during an ophthalmic surgical procedure, comprising, a fluid source coupled with a surgical console having a first volume of fluid, an irrigation pump fluidly coupled with the fluid source and in communication with an encoder for tracking the activation time of the irrigation pump, and an instrument host for receiving and applying a conversion factor to the activation time to provide a second volume of fluid, wherein the second volume of fluid subtracted from the first volume of fluid provides an indication of the amount of fluid remaining in the fluid source.

### Example 46

The method of example 45, wherein each state indication is indicative of an open or closed valve position.

### Example 47

The method of any one of examples 45 to 46, wherein the encoder tracks activity of a drainage pump that is in communication with the cassette.

### Example 48

The method of any one of examples 45 to 47, wherein the activity of the drainage pump is selected from the group consisting of pump runtime, pump speed, and direction.

### Example 49

The method of any one of examples 45 to 48, wherein the calculating the amount of fluid removed from the fluid source further includes summing the amount of fluid associated with the encoder tracked activity of the drainage pump.

### Example 50

The method of any one of examples 45 to 49, wherein the encoder tracks activity of an irrigation pump that is in communication with the cassette.

### Example 51

The method of example 50, wherein the calculating the amount of fluid removed from the fluid source further includes summing the amount of fluid associated with the encoder tracked activity of the irrigation pump.

### Example 52

The method of any one of examples 45 to 51, wherein the calculating the amount of fluid removed from the fluid source further includes summing the amount of fluid associated with wound leakage.

### Example 53

The method of any one of examples 45 to 52, wherein the fluid capacity of the cassette includes a handpiece volume and an aspiration and irrigation tubing.

### Example 54

The method of any one of examples 45 to 53, wherein the volume of the fluid source is an initial size of the fluid source coupled with the surgical console.

### Example 55

The method of any one of examples 45 to 53, wherein the volume of the fluid source is carried over from a previous ophthalmic surgical procedure.

### Example 56

The method of any one of examples 45 to 54, further comprising providing a warning when the amount of fluid remaining in the fluid source drops below a threshold value.

### Example 57

A method for monitoring the amount of fluid used during an ophthalmic surgical procedure, comprising, starting a surgical procedure with a known volume of an irrigation fluid in a fluid source, administering the irrigation fluid; starting a timer, calculating an accumulated irrigation flow rate, stopping the administering of irrigation fluid and the timer, acquiring a total elapsed time, and calculating a total amount of irrigation fluid used during the surgical procedure.

### Example 58

The method of example 57, wherein the accumulated irrigation flow is sum of a positive irrigation and negative irrigation.

### Example 59

The method of any one of examples 57 to 58, wherein the amount of irrigation fluid used during the surgical procedure is determined by Equation 1.

### Example 60

The method of any one of examples 57 to 59, wherein the known volume of irrigation fluid is carried over from a previous surgical procedure.

### Example 61

The method of any one of examples 57 to 59, wherein the known volume of irrigation fluid may be inputted by a user.

### Example 62

The method of any one of examples 57 to 61, wherein the method further comprises providing a warning when the amount of fluid remaining in the fluid source drops below a threshold value.

### Example 63

A system for monitoring the amount of fluid used during an ophthalmic surgical procedure, comprising: a fluid source coupled with a surgical console having a known volume of irrigation fluid; an irrigation pump fluidly coupled with the fluid source and in communication with an encoder for tracking the activation time of the irrigation pump; and an instrument host configured to: start a surgical procedure with the known volume of irrigation fluid in the fluid source; administer irrigation fluid; start a timer; calculate an accumulated irrigation flow rate; stop the administering of irrigation fluid and the timer; acquire a total elapsed time; and calculate a total amount of irrigation fluid used during the surgical procedure.

### Example 64

The system of example 63, wherein the accumulated irrigation flow is a sum of a positive irrigation and negative irrigation.

### Example 65

The system of any one of examples 63 to 64, wherein the amount of irrigation fluid used during the surgical procedure is determined by the following formula:

$$Solution Used = ( ( \frac{Accumulated Irrigation Flow}{\begin{matrix}Count of Total Number \\ of Accumulated Values\end{matrix}} ) \times Elapsed Time ) \div 60$$

### Example 66

The system of any one of examples 63 to 65, wherein the known volume of irrigation fluid is carried over from a previous surgical procedure.

### Example 67

The system of any one of examples 63 to 66, wherein the known volume of the irrigation fluid is inputted by a user.

### Example 68

The system of any one of examples 63 to 67, further comprising providing a warning when the amount of fluid remaining in the fluid source drops below a threshold value.

Those of skill in the art will appreciate that the herein described apparatuses, engines, devices, systems, and methods are susceptible to various modifications and alternative constructions. There is no intention to limit the scope of the aspect of the disclosure to the specific constructions described herein. Rather, the herein described systems and methods are intended to cover all modifications, alternative constructions, and equivalents falling within the scope and spirit of the disclosure, any appended claims and any equivalents thereto.

In the foregoing detailed description, it may be that various features are grouped together in individual examples for the purpose of brevity in the disclosure. This method of disclosure is not to be interpreted as reflecting an intention that any subsequently claimed examples require more features than are expressly recited.

Further, the descriptions of the disclosure are provided to enable any person skilled in the art to make or use the disclosed examples. Various modifications to the disclosure will be readily apparent to those skilled in the art, and the generic principles defined herein may be applied to other variations without departing from the spirit or scope of the disclosure. Thus, the disclosure is not intended to be limited to the examples and designs described herein, but rather is to be accorded the widest scope consistent with the principles and novel features disclosed herein.

1-43. (canceled)

44. A method for monitoring the amount of fluid used during an ophthalmic surgical procedure, comprising:
inputting into a surgical console the volume of a fluid source in communication with the surgical console;
attaching to the surgical console a cassette having a known fluid capacity;
receiving by the surgical console an encoder value for tracking the use of at least one pump and a plurality of state indications of an irrigation valve control; and
calculating the amount of fluid removed from the fluid source by summing the plurality of state indications and the fluid capacity of the cassette.

45. The method of claim 44, wherein each state indication is indicative of an open or closed valve position.

46. The method of claim 44, wherein the encoder tracks activity of a drainage pump that is in communication with the cassette.

47. The method of claim 46, wherein the activity of the drainage pump is selected from the group consisting of pump runtime, pump speed, and direction.

48. The method of claim 46, wherein the calculating the amount of fluid removed from the fluid source further includes summing the amount of fluid associated with the encoder tracked activity of the drainage pump.

49. The method of claim 44, wherein the encoder tracks activity of an irrigation pump that is in communication with the cassette.

50. The method of claim 49, wherein the calculating the amount of fluid removed from the fluid source further includes summing the amount of fluid associated with the encoder tracked activity of the irrigation pump.

51. The method of claim 44, wherein the calculating the amount of fluid removed from the fluid source further includes summing the amount of fluid associated with wound leakage.

52. The method of claim 44, wherein the fluid capacity of the cassette includes a handpiece volume and an aspiration and irrigation tubing.

53. The method of claim 44, wherein the volume of the fluid source is an initial size of the fluid source coupled with the surgical console.

54. The method of claim 44, wherein the volume of the fluid source is carried over from a previous ophthalmic surgical procedure.

55. The method of claim 44, further comprising providing a warning when the amount of fluid remaining in the fluid source drops below a threshold value.

56. A method for monitoring the amount of fluid used or remaining in a fluid source during an ophthalmic surgical procedure, comprising:
starting a surgical procedure with a known volume of an irrigation fluid in a fluid source;
administering the irrigation fluid;
starting a timer;
calculating an accumulated irrigation flow rate;
stopping the administering of irrigation fluid and the timer;
acquiring a total elapsed time; and
calculating a total amount of irrigation fluid used during the surgical procedure.

57. The method of claim 56, wherein the accumulated irrigation flow is a sum of a positive irrigation and negative irrigation.

58. The method of claim 56, wherein the amount of irrigation fluid used during the surgical procedure is determined by the following formula:
$$Solution Used = ( ( \frac{Accumulated Irrigation Flow}{\begin{matrix}Count of Total Number \\ of Accumulated Values\end{matrix}} ) \times Elapsed Time ) \div 60$$

59. The method of claim 56, wherein the known volume of irrigation fluid is carried over from a previous surgical procedure.

60. The method of claim 56, wherein the known volume of the irrigation fluid is inputted by a user.

61. The method of claim 56, further comprising providing a warning when the amount of fluid remaining in the fluid source drops below a threshold value.

62. A system for monitoring the amount of fluid used during an ophthalmic surgical procedure, comprising:
a fluid source coupled with a surgical console having a known volume of irrigation fluid;
an irrigation pump fluidly coupled with the fluid source and in communication with an encoder for tracking the activation time of the irrigation pump; and
an instrument host configured to:
start a surgical procedure with the known volume of irrigation fluid in the fluid source;
administer irrigation fluid;
start a timer;
calculate an accumulated irrigation flow rate;
stop the administering of irrigation fluid and the timer;
acquire a total elapsed time; and
calculate a total amount of irrigation fluid used during the surgical procedure.

63. The system of claim 62, wherein the accumulated irrigation flow is a sum of a positive irrigation and negative irrigation.

64. The system of claim 62, wherein the amount of irrigation fluid used during the surgical procedure is determined by the following formula:
$$Solution Used = ( ( \frac{Accumulated Irrigation Flow}{\begin{matrix}Count of Total Number \\ of Accumulated Values\end{matrix}} ) \times Elapsed Time ) \div 60$$

65. The system of claim 62, wherein the known volume of irrigation fluid is carried over from a previous surgical procedure.

66. The system of claim 62, wherein the known volume of the irrigation fluid is inputted by a user.

67. The system of claim 62, further comprising providing a warning when the amount of fluid remaining in the fluid source drops below a threshold value.
